(19) United States
(12) Patent Application Publication
(10) Pub. No.: US 2003/0138808 A1
(43) Pub. Date: Jul. 24, 2003

(54) Expression vectors encoding epitopes of target-associated antigens

(76) Inventors: John J.L. Simard, Vancouver (CA); Xiang-Dong Lei, West Hills, CA (US); David C. Diamond, West Hills, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/225,568

(22) Filed: Aug. 20, 2002

Related U.S. Application Data
(63) Continuation-in-part of application No. 09/561,572, filed on Apr. 28, 2000.
(63) Continuation-in-part of application No. 09/380,534, filed on Sep. 1, 1999.
(63) Continuation-in-part of application No. 09/776,232, filed on Feb. 2, 2001.
(63) Continuation-in-part of application No. 09/715,835, filed on Nov. 16, 2000.
(63) Continuation-in-part of application No. 09/026,066, filed on Feb. 19, 1998, now Pat. No. 6,067,866.
(63) Continuation-in-part of application No. 10/117,937, filed on Apr. 4, 2002.
(60) Provisional application No. 60/337,017, filed on Nov. 7, 2001.
(60) Provisional application No. 60/336,968, filed on Nov. 7, 2001.
(60) Provisional application No. 60/363,210, filed on Mar. 7, 2002.

(51) Int. Cl. C12Q 1/68; C07H 21/04

(52) U.S. Cl. 435/6; 536/23.2

(57) ABSTRACT

The invention provides a nucleic acid construct encoding a housekeeping epitope derived from an antigen associated with a target cell. The construct can further include a second sequence encoding a housekeeping epitope or an immune epitope. The construct can be used as a vaccine against numerous kinds of target cells, including neoplastic cells and cells infected with an intracellular virus.

### CROSS REFERENCE TO RELATED APPLICATIONS

This application is a Continuation-in-Part of and claims priority from U.S. patent application Ser. No. 09/561,572, entitled “EXPRESSION VECTORS ENCODING EPITOPES OF TARGET-ASSOCIATED ANTIGENS,” filed on Apr. 28, 2001; application Ser. No. 09/380,534, entitled “A METHOD OF INDUCING A CTL RESPONSE,” filed on Sep. 1, 1999; application Ser. No. 09/776,232, entitled “A METHOD OF INDUCING A CTL RESPONSE,” filed on Feb. 2, 2001; application Ser. No. 09/715,835, entitled “AVOIDANCE OF UNDESIRABLE REPLICATION INTERMEDIATES IN PLASMID PROPAGATION,” filed on Nov. 16, 2000; Application No. 60/337,017, entitled “EPITOPE SEQUENCES,” filed on Nov. 7, 2001; Application No. 60/336,968, entitled “EXPRESSION VECTORS ENCODING EPITOPES OF TARGET-ASSOCIATED ANTIGENS AND METHODS FOR THEIR DESIGN,” filed on Nov. 7, 2001; application Ser. No. 09/026,066, entitled “EPITOPE SYNCHRONIZATION IN ANTIGEN PRESENTING CELLS,” filed on Dec. 7, 2001; Application No. 60/363,210, entitled “EPITOPE SEQUENCES,” filed on Mar. 7, 2002; and Application No. 10/117,937, entitled “EPITOPE SEQUENCES,” filed on Apr. 4, 2002. Each of the above-mentioned priority applications is hereby incorporated by reference in its entirety.

### BACKGROUND OF THE INVENTION

1. Field of the Invention

The invention disclosed herein is directed to epitope-encoding vectors for use in pharmaceutical compositions capable of inducing an immune response in a subject to whom the compositions are administered. The epitopes expressed using such vectors can stimulate a cellular immune response against a target cell displaying the epitope(s).

2. Description of the Related Art

Neoplasia and the Immune System

The neoplastic disease state commonly known as cancer is thought to generally result from a single cell growing out of control. The uncontrolled growth state typically results from a multi-step process in which a series of cellular systems fail, resulting in the genesis of a neoplastic cell. The resulting neoplastic cell rapidly reproduces itself, forms one or more tumors, and eventually may cause the death of the host.

Because the progenitor of the neoplastic cell shares the host's genetic material, neoplastic cells are largely exempt from the host's immune system. During immune surveillance, the process in which the host's immune system surveys and localizes foreign materials, a neoplastic cell will appear to the host's immune surveillance machinery as a “self” cell.

Viruses and the Immune System

In contrast to cancer cells, virus infection involves the expression of clearly non-self antigens. As a result, many virus infections are successfully dealt with by the immune system with minimal clinical sequela. Moreover, it has been possible to develop effective vaccines for many of those infections that do cause serious disease. A variety of vaccine approaches have been successfully used to combat various diseases. These approaches include subunit vaccines consisting of individual proteins produced through recombinant DNA technology. Notwithstanding these advances, the selection and effective administration of minimal epitopes for use as viral vaccines has remained problematic.

In addition to the difficulties involved in epitope selection stands the problem of viruses that have evolved the capability of evading a host's immune system. Many viruses, especially viruses that establish persistent infections, such as members of the herpes and pox virus families, produce immunomodulatory molecules that permit the virus to evade the host's immune system. The effects of these immunomodulatory molecules on antigen presentation may be overcome by the targeting of select epitopes for administration as immunogenic compositions. To better understand the interaction of neoplastic cells and virally infected cells with the host's immune system, a discussion of the system's components follows below.

The immune system functions to discriminate molecules endogenous to an organism (“self” molecules) from material exogenous or foreign to the organism (“non-self” molecules). The immune system has two types of adaptive responses to foreign bodies based on the components that mediate the response: a humoral response and a cell-mediated response. The humoral response is mediated by antibodies, while the cell-mediated response involves cells classified as lymphocytes. Recent anticancer and antiviral strategies have focused on mobilizing the host immune system as a means of anticancer or antiviral treatment or therapy.

The immune system functions in three phases to protect the host from foreign bodies: the cognitive phase, the activation phase, and the effector phase. In the cognitive phase, the immune system recognizes and signals the presence of a foreign antigen or invader in the body. The foreign antigen can be, for example, a cell surface marker from a neoplastic cell or a viral protein. Once the system is aware of an invading body, antigen specific cells of the immune system proliferate and differentiate in response to the invader-triggered signals. The last stage is the effector stage in which the effector cells of the immune system respond to and neutralize the detected invader.

An array of effector cells implement an immune response to an invader. One type of effector cell, the B cell, generates antibodies targeted against foreign antigens encountered by the host. In combination with the complement system, antibodies direct the destruction of cells or organisms bearing the targeted antigen. Another type of effector cell is the natural killer cell (NK cell), a type of lymphocyte having the capacity to spontaneously recognize and destroy a variety of virus infected cells as well as malignant cell types. The method used by NK cells to recognize target cells is poorly understood.

Another type of effector cell, the T cell, has members classified into three subcategories, each playing a different role in the immune response. Helper T cells secrete cytokines which stimulate the proliferation of other cells necessary for mounting an effective immune response, while suppressor T cells down-regulate the immune response. A third category of T cell, the cytotoxic T cell (CTL), is capable of directly lysing a targeted cell presenting a foreign antigen on its surface.

The Major Histocompatibility Complex and T Cell Target Recognition

T cells are antigen specific immune cells that function in response to specific antigen signals. B lymphocytes and the antibodies they produce are also antigen specific entities. However, unlike B lymphocytes, T cells do not respond to antigens in a free or soluble form. For a T cell to respond to an antigen, it requires the antigen to be bound to a presenting complex known as the major histocompatibility complex (MHC).

MHC complex proteins provide the means by which T cells differentiate native or “self” cells from foreign cells. There are two types of MHC, class I MHC and class 1I MHC. T Helper cells (CD4⁺) predominately interact with class II MHC proteins while cytolytic T cells (CD8⁺) predominately interact with class I MHC proteins. Both MHC complexes are transmembrane proteins with a majority of their structure on the external surface of the cell. Additionally, both classes of MHC have a peptide binding cleft on their external portions. It is in this cleft that small fragments of proteins, native or foreign, are bound and presented to the extracellular enviroment.

Cells called antigen presenting cells (APCs) display antigens to T cells using the MHC complexes. For T cells to recognize an antigen, it must be presented on the MHC complex for recognition. This requirement is called MHC restriction and it is the mechanism by which T cells differentiate “self” from “non-self” cells. If an antigen is not displayed by a recognizable MHC complex, the T cell will not recognize and act on the antigen signal. T cells specific for the peptide bound to a recognizable MHC complex bind to these MHC-peptide complexes and proceed to the next stages of the immune response.

As discussed above, neoplastic cells are largely ignored by the immune system. A great deal of effort is now being expended in an attempt to harness a host's immune system to aid in combating the presence of neoplastic cells in a host. One such area of research involves the formulation of anticancer vaccines.

Anticancer Vaccines

Among the various weapons available to an oncologist in the battle against cancer is the immune system of the patient. Work has been done in various attempts to cause the immune system to combat cancer or neoplastic diseases. Unfortunately, the results to date have been largely disappointing. One area of particular interest involves the generation and use of anticancer vaccines.

To generate a vaccine or other immunogenic composition, it is necessary to introduce to a subject an antigen or epitope against which an immune response may be mounted. Although neoplastic cells are derived from and therefore are substantially identical to normal cells on a genetic level, many neoplastic cells are known to present tumor-associated antigens (TAAs). In theory, these antigens could be used by a subject's immune system to recognize these antigens and attack the neoplastic cells. Unfortunately, neoplastic cells appear to be ignored by the host's immune system.

A number of different strategies have been developed in an attempt to generate vaccines with activity against neoplastic cells. These strategies include the use of tumor associated antigens as immunogens. For example, U.S. Pat. No. 5,993,828, describes a method for producing an immune response against a particular subunit of the Urinary Tumor Associated Antigen by administering to a subject an effective dose of a composition comprising inactivated tumor cells having the Urinary Tumor Associated Antigen on the cell surface and at least one tumor associated antigen selected from the group consisting of GM-2, GD-2, Fetal Antigen and Melanoma Associated Antigen. Accordingly, this patent describes using whole, inactivated tumor cells as the immunogen in an anticancer vaccine.

Another strategy used with anticancer vaccines involves administering a composition containing isolated tumor antigens. In one approach, MAGE-A1 antigenic peptides were used as an immunogen. (See Chaux, P., et al., “Identification of Five MAGE-A1 Epitopes Recognized by Cytolytic T Lymphocytes Obtained by In Vitro Stimulation with Dendritic Cells Transduced with MAGE-A1,” J. Immunol., 163(5):2928-2936 (1999)). There have been several therapeutic trials using MAGE-A1 peptides for vaccination, although the effectiveness of the vaccination regimes was limited. The results of some of these trials are discussed in Vose, J. M., “Tumor Antigens Recognized by T Lymphocytes,” 10^{th} European Cancer Conference, Day 2, Sep. 14, 1999.

In another example of tumor associated antigens used as vaccines, Scheinberg, et al. treated 12 chronic myclogenous leukemia (CML) patients already receiving interferon (IFN) or hydroxyurea with 5 injections of class I-associated bcr-abl peptides with a helper peptide plus the adjuvant QS-21. Scheinberg, D. A., et al., “BCR-ABL Breakpoint Derived Oncogene Fusion Peptide Vaccines Generate Specific Immune Responses in Patients with Chronic Myelogenous Leukemia (CML) [Abstract 1665], American Society of Clinical Oncology 35^{th} Annual Meeting, Atlanta (1999). Proliferative and delayed type hypersensitivity (DTH) T cell responses indicative of T-helper activity were elicited, but no cytolytic killer T cell activity was observed within the fresh blood samples.

Additional examples of attempts to identify TAAs for use as vaccines are seen in the recent work of Cebon, et al. and Scheibenbogen, et al. Cebon et al. Immunized patients with metastatic melanoma using intradermallly administered MART-1₂₆₋₃₅ peptide with IL-12 in increasing doses given either subcutaneously or intravenously. Of the first 15 patients, 1 complete remission, 1 partial remission, and 1 mixed response were noted. Immune assays for T cell generation included DTH, which was seen in patients with or without IL-12. Positive CTL assays were seen in patients with evidence of clinical benefit, but not in patients without tumor regression. Cebon, et al., “Phase I Studies of Immunization with Melan-A and IL-12 in HLA A2+Positive Patients with Stage III and IV Malignant Melanoma,” [Abstract 1671], American Society of Clinical Oncology 35^{th} Annual Meeting, Atlanta (1999).

Scheibenbogen, et al. immunized 18 patients with 4 HLA class I restricted tyrosinase peptides, 16 with metastatic melanoma and 2 adjuvant patients. Scheibenbogen, et al., “Vaccination with Tyrosinase peptides and GM-CSF in Metastatic Melanoma: a Phase II Trial,” [Abstract 1680], American Society of Clinical Oncology 35^{th} Annual Meeting, Atlanta (1999). Increased CTL activity was observed in 4/15 patients, 2 adjuvant patients, and 2 patients with evidence of tumor regression. As in the trial by Cebon et al., patients with progressive disease did not show boosted immunity. In spite of the various efforts expended to date to generate efficacious anticancer vaccines, no such composition has yet been developed.

Vaccine strategies to protect against viral diseases have had many successes. Perhaps the most notable of these is the progress that has been made against the disease small pox, which has been driven to extinction. The success of the polio vaccine is of a similar magnitude.

Viral vaccines can be grouped into three classifications: live attenuated virus vaccines, such as vaccinia for small pox, the Sabin poliovirus vaccine, and measles mumps and rubella; whole killed or inactivated virus vaccines, such as the Salk poliovirus vaccine, hepatitis A virus vaccine and the typical influenza virus vaccines; and subunit vaccines, such as hepatitis B. Due to their lack of a complete viral genome, subunit vaccines offer a greater degree of safety than those based on whole viruses.

The paradigm of a successful subunit vaccine is the recombinant hepatitis B vaccine based on the viruses envelope protein. Despite much academic interest in pushing the subunit concept beyond single proteins to individual epitopes the efforts have yet to bear much fruit. Viral vaccine research has also concentrated on the induction of an antibody response although cellular responses also occur. However, many of the subunit formulations are particularly poor at generating a CTL response.

### SUMMARY OF THE INVENTION

The invention provides a nucleic acid construct including a first coding region, wherein the first coding region includes a first sequence encoding at least a first polypeptide, wherein the first polypeptide includes a first housekeeping epitope derived from a first antigen associated with a first target cell. The first coding region can further include a second sequence encoding at least a second polypeptide, wherein the second polypeptide includes an second epitope derived from a second antigen associated with a second target cell. The first polypeptide and the second polypeptide can contiguous or non-contiguous. The second epitope can be a housekeeping epitope or an immune epitope. The first antigen and the second antigen can be the same or different; likewise, the first and second target cells can be the same or different.

The target cell can be a neoplastic cell, such as, for example, leukemia, carcinoma, lymphoma, astrocytoma, sarcoma, glioma, retinoblastoma, melanoma, Wilm's tumor, bladder cancer, breast cancer, colon cancer, hepatocellular cancer, pancreatic cancer, prostate cancer, lung cancer, liver cancer, stomach cancer, cervical cancer, testicular cancer, renal cell cancer, or brain cancer. The first antigen can be, for example, MART-1/MelanA, gp100 (Pmel 17), tyrosinase, TRP-1, TRP-2, MAGE-1, MAGE-3, BAGE, GAGE-1, GAGE-2, pl5, NY-ESO, products of an SSX gene family member, CT-7, and products of an SCP gene family member. The target cell can be infected by a virus such as, for example, adenovirus, cytomegalovirus, Epstein-Barr virus, herpes simplex virus 1 and 2, human herpesvirus 6, varicella-zoster virus, hepatitis B virus, hepatitis D virus, papillomavirus, parvovirus B19, polyomavirus BK, polyomavirus JC, hepatitis C virus, measles virus, rubella virus, human immunodeficiency virus (HIV), human T-cell leukemia virus I, or human T-cell leukemia virus II. The target cell can likewise be infected by a bacterium, a protozoan, a fungus, a prion, or any other intracellular parasite, examples of which are Chlamydia, Listeria, Salmonella, Legionella, Brucella, Coxiella, Rickettsia, Mycobacterium, Leishmania, Trypanasoma, Toxoplasma, and Plasmodium.

The construct typically includes a first promoter sequence operably linked to the first coding region. The promoter can be, for example, cytomegalovirus (CMV), SV40 and retroviral long terminal repeat (LTR). The promoter can be a bidirectional promoter, and/or a second promoter sequence can be operably linked to a second coding region. The nucleic acid construct can further include a poly-A sequence operably linked to the first coding region, the second coding region, or both. The nucleic acid construct can also include an internal ribosome entry site (IRES) sequence, a ubiquitin sequence, an autocatalytic peptide sequence, enhancers, nuclear import sequences, immunostimulatory sequences, and expression cassettes for cytokines, selection markers, reporter molecules, and the like. The first polypeptide can be about 7 to 15 amino acids in length, and is preferably 9 or 10 amino acids in length. The second polypeptide can be 9 or 10 amino acids in length, or it can be an epitope cluster between about 10 and about 75 amino acids in length. The first epitope and second epitopes can bind the same or different alleles of MHC.

Other embodiments of the invention include a vaccine that includes any of the foregoing nucleic acid construct embodiments; a method of treating an animal by administering such a vaccine; and a method of making the vaccine.

Still further embodiments relate to nucleic acid constructs that include a first coding region, wherein the first coding region includes a first sequence encoding a first polypeptide, wherein the first polypeptide includes a first housekeeping epitope corresponding to a housekeeping proteasome cleavage product of tyrosinase, wherein the mature/fully-processed housekeeping epitope is an MHC epitope has a sequence and the sequence is, for example, SEQ ID NO. 5, a sequence with functional similarity to SEQ ID NO. 5, a sequence with substantial similarity to SEQ ID NO. 5, and the like. The MHC epitope can have the sequence of SEQ ID NO. 5. This construct can be used in an immunogenic composition, for example.

The sequence of the first polypeptide can be, for example, SEQ ID NO. 5, SEQ ID NO. 6, a sequence with functional similarity to SEQ ID NO. 5, a sequence with functional similarity to SEQ ID NO. 6, a sequence with substantial similarity to SEQ ID NO. 5, a sequence with substantial similarity to SEQ ID NO. 6, and the like. More preferably, the first polypeptide can have the sequence of SEQ ID NO. 5, SEQ ID NO. 6, or any other like sequence, for example.

Embodiments of the invention also relate to nucleic acid constructs that include a first coding region, wherein the first coding region includes a first sequence encoding a first polypeptide, wherein the first polypeptide includes a first housekeeping epitope corresponding to a housekeeping proteasome cleavage product of a first antigen associated with a melanoma cell, wherein the mature/fully-processed housekeeping epitope is an MHC epitope, wherein the first coding region further includes a second sequence encoding a second polypeptide, wherein the second polypeptide includes an epitope cluster derived from tyrosinase. This construct can be used in an immunogenic composition, for example.

The first coding region and the second coding region can be transcribed as segments of a single transcript, joined by an IRES, for example. The sequence of the epitope cluster can be, for example, SEQ ID NO. 7, a sequence with functional similarity to SEQ ID NO. 7, a sequence with substantial similarity to SEQ ID NO. 7, and the like. More preferably, epitope cluster includes the sequence of SEQ ID NO. 7.

The mature/fully-processed housekeeping epitope can be an MHC epitope, and the sequence can be, for example, SEQ ID NO. 5, a sequence with functional similarity to SEQ ID NO. 5, a sequence with substantial similarity to SEQ ID NO. 5, and the like. Preferably, the MHC epitope includes the sequence of SEQ ID NO. 5.

Further embodiments of the invention relate to nucleic acid constructs that include a sequence, such as, for example, the sequence of SEQ ID NO. 8, a sequence with functional similarity to SEQ ID NO. 8, a sequence with substantial similarity to SEQ ID NO. 8, and the like. Preferably, constructs include the sequence of SEQ ID NO. 8. This construct can be used in an immunogenic composition, for example.

Other embodiments of the invention relate to nucleic acid constructs that include a first coding region, wherein the first coding region includes a first sequence encoding a first polypeptide, wherein the first polypeptide includes a first housekeeping epitope corresponding to a housekeeping proteasome cleavage product of a first antigen associated with a melanoma cell, wherein the mature/fully-processed housekeeping epitope is an MHC epitope. The nucleic acid constructs can further include a second coding region that includes a second sequence encoding a second polypeptide, wherein the second polypeptide includes an epitope cluster derived from tyrosinase. This construct can be used in an immunogenic composition, for example.

The epitope cluster can have a sequence, and for example, the sequence can be SEQ ID NO. 7, a sequence with functional similarity to SEQ ID NO. 7, a sequence with substantial similarity to SEQ ID NO. 7, and the like. The mature/fully-processed housekeeping epitope can be an MHC epitope having a sequence, and the sequence can be, for example, SEQ ID NO. 5, a sequence with functional similarity to SEQ ID NO. 5, a sequence with substantial similarity to SEQ ID NO. 5, and the like.

Still further embodiments of the invention relate to immunogenic compositions that include any of the nucleic acid constructs of the embodiments described above as well as any others described herein. Other embodiments relate to methods of treating using the immunogenic compositions and to methods of making the same.

### BRIEF DESCRIPTION OF THE DRAWINGS

FIG. 1 is a depiction of the components of plasmid pVAX-EP1-IRES-EP2-ISS-NIS.

FIG. 2 is a depiction of the components of plasmid pVAX-EP2-UB-EP1.

FIG. 3 is a depiction of the components of plasmid pVAX-EP2-2A-EP1.

FIG. 4 is a depiction of the components of plasmid pVAX-EP1-IRES-EP2.

FIG. 5 displays the locations of the IRES and the encoded polypeptides, with the translations of the polypeptides (SEQ ID NO. 8).

FIG. 6 shows the insertion of a cannula into inguinal lymph node under ultrasound guidance.

FIG. 7 graphically shows the results of a tetramer assay on fresh blood to tyrosinase pre- and post-vaccine. Tetramer positive cells as a percent of total CD8 positive cells is shown on the ordinate, with the pre-vaccine, 2 and 4 weeks and post-vaccine time points. Patients were grouped by dose on the abscissa.

FIG. 8 shows survival results. Survival is plotted for evaluable patients with percentage of patients alive on the ordinate and time in weeks on the abscissa. FIG. 8A demonstrates overall survival for all evaluable patients. FIG. 8B demonstrates survival for all evaluable patients separated by immune response.

### DETAILED DESCRIPTION OF THE PREFERRED EMBODIMENT

Aspects of the present invention provide nucleic acid constructs that encode a housekeeping epitope. A housekeeping epitope, as will be described in greater detail below, includes peptide fragments produced by the active proteasome of a peripheral cell. A basis for the present invention is the discovery that any antigen associated with a target cell can be processed differentially into two distinguishable sets of epitopes for presentation by the class I major histocompatibility complex (MHC) molecules of the body. “Immune epitopes” are presented by pAPCs and, also generally in peripheral cells that are acutely infected or under active immunological attack by interferon (IFN) secreting cells. In contrast, “housekeeping epitopes” are presented by all other peripheral cells including, generally, neoplastic (cancerous) cells and chronically infected cells. This mismatch, or asynchrony, in presented epitopes underlies the persistence and advance of cancers and chronic infections, despite the presence of a functioning immune system in the host. It is thus essential to bring about synchronization of epitope presentation between the pAPC and the target cell in order to provoke an effective, cytolytic T lymphocyte (CTL)-mediated immune response. Epitope synchronization, and vaccines and treatments based on epitope synchronization, are described in detail in copending U.S. patent application Ser. No. 09/560,465 entitled “EPITOPE SYNCHRONIZATION IN ANTIGEN PRESENTING CELLS,” filed on Apr. 28, 2000, which is incorporated herein by reference in its entirety.

Synchronization can be accomplished most reliably by providing the pAPC with a housekeeping epitope. Often a more robust response can be achieved by providing more than a single epitope. Additionally, once an effective immune response against the target cells has been established, secretion of IFN may lead to expression of the immune proteasome, thereby switching epitope presentation to immune epitopes. For this reason, among others, it can also be advantageous to include immune epitopes, in addition to housekeeping epitopes, in vaccines developed according to the above referenced disclosure. It can be of further utility to provide immune epitopes in the form of an epitope cluster region as defined in copending U.S. patent application Ser. No. 09/561,571 entitled “EPITOPE CLUSTERS,” filed on Apr. 28, 2000, which is incorporated herein by reference in its entirety. Embodiments of the invention provide expression vectors encoding housekeeping epitopes and/or immune epitopes in a variety of combinations. Preferred expression constructs encode at least one epitope capable of stimulating a cellular immune response directed against a target cell. In one embodiment of the invention, target cells are neoplastic cells. In another embodiment, target cells are any intracellularly infected host cell. Intracellular infective agents include persistent viruses and any other infectious organism that has an intracellular stage of infection.

The nucleic acid constructs of some embodiments are directed to enhancing a subject's immune system and sensitizing it to the presence of neoplastic cells within the host. In other embodiments, the nucleic acid constructs facilitate the eradication of persistent viral infections as well as cells infected with intracellular parasites.

Definitions

Unless-otherwise clear from the context of the use of a term herein, the following listed terms shall generally have the indicated meanings for purposes of this description.

PROFESSIONAL ANTIGEN-PRESENTING CELL (pAPC)—a cell that possesses T cell costimulatory molecules and is able to induce a T cell response. Well characterized pAPCs are dendritic cells, B cells, and macrophages.

PERIPHERAL CELL—a cell that is not a pAPC.

HOUSEKEEPING PROTEASOME—a proteasome normally active in peripheral cells, and generally not present or not strongly active in pAPCs.

IMMUNE PROTEASOME—a proteasome normally active in pAPCs; the immune proteasome is also active in some peripheral cells in infected tissues.

EPITOPE—a molecule or substance capable of stimulating an immune response. In preferred embodiments, epitopes according to this definition include but are not necessarily limited to a polypeptide and a nucleic acid encoding a polypeptide, wherein the polypeptide is capable of stimulating an immune response. In other preferred embodiments, epitopes according to this definition include but are not necessarily limited to peptides presented on the surface of cells non-covalently bound to the pocket of class I MHC, such that they can interact with T cell receptors.

MHC EPITOPE—a polypeptide having a known or predicted affinity for a mammalian class I major histocompatibility complex (MHC) molecule.

HOUSEKEEPING EPITOPE—In a preferred embodiment, a housekeeping epitope is defined as a polypeptide fragment that is an MHC epitope, and that is displayed on a cell in which housekeeping proteasomes are predominantly active. In another preferred embodiment, a housekeeping epitope is defined as a polypeptide containing a housekeeping epitope according to the foregoing definition, that is flanked by one to several additional amino acids. In another preferred embodiment, a housekeeping epitope is defined as a nucleic acid that encodes a housekeeping epitope according to either of the foregoing definitions.

IMMUNE EPITOPE—In a preferred embodiment, an immune epitope is defined as a polypeptide fragment that is an MHC epitope, and that is displayed on a cell in which immune proteasomes are predominantly active. In another preferred embodiment, an immune epitope is defined as a polypeptide containing an immune epitope according to the foregoing definition, that is flanked by one to several additional amino acids. In another preferred embodiment, an immune epitope is defined as a polypeptide including an epitope cluster sequence, having at least two polypeptide sequences having a known or predicted affinity for a class I MHC. In yet another preferred embodiment, an immune epitope is defined as a nucleic acid that encodes an immune epitope according to any of the foregoing definitions.

TARGET CELL—a cell to be targeted by the vaccines and methods of the invention. Examples of target cells according to this definition include but are not necessarily limited to: a neoplastic cell and a cell harboring an intracellular parasite, such as, for example, a virus, a bacterium, or a protozoan.

TARGET-ASSOCIATED ANTIGEN (TAA)—a protein or polypeptide present in a target cell.

TUMOR-ASSOCIATED ANTIGENS (TuAA)—a TAA, wherein the target cell is a neoplastic cell.

ENCODE—an open-ended term such that a nucleic acid encoding a particular amino acid sequence can consist of codons specifying that (poly)peptide, but the nucleic acid can also comprise additional sequences, either translatable, or for the control of transcription, translation, or replication, or to facilitate manipulation of some host nucleic acid construct.

SUBSTANTIAL SIMILARITY—this term is used to refer to sequences that differ from a reference sequence in an inconsequential way as judged by examination of the sequence. Nucleic acid sequences encoding the same amino acid sequence are substantially similar despite differences in degenerate positions or modest differences in length or composition of any non-coding regions. Amino acid sequences differing only by conservative substitution or minor length variations are substantially similar. Additionally, amino acid sequences comprising housekeeping epitopes that differ in the number of N-terminal flanking residues, or immune epitopes and epitope clusters that differ in the number of flanking residues at either terminus, are substantially similar. Nucleic acids that encode substantially similar amino acid sequences are themselves also substantially similar.

FUNCTIONAL SIMILARITY—this term is used to refer to sequences that differ from a reference sequence in an inconsequential way as judged by examination of a biological or biochemical property, although the sequences may not be substantially similar. For example, two nucleic acids can be useful as hybridization probes for the same sequence but encode differing amino acid sequences. Two peptides that induce cross-reactive CTL responses are functionally similar even if they differ by non-conservative amino acid substitutions (and thus do not meet the substantial similarity definition). Pairs of antibodies, or TCRs, that recognize the same epitope can be functionally similar to each other despite whatever structural differences exist. Functional similarity of immunogenicity can be confirmed, for example, by immunizing with the “altered” antigen and testing the ability of the elicited response (Ab, CTL, cytokine production, etc.) to recognize the target antigen. Accordingly, two sequences may be designed to differ in certain respects while retaining the same function. Such designed sequence variants are among the embodiments of the present invention.

MATURE HOUSEKEEPING EPITOPE—this term refers to an MHC epitope in distinction to any precursor that may consist essentially of a housekeeping epitope, but also includes other sequences in a primary translation product that are removed by processing, including without limitation, alone or in any combination proteasomal digestion, N-terminal trimming, or the action of exogenous enzymatic activities.

CONSISTING ESSENTIALLY OF A HOUSEKEEPING EPITOPE—a sequence consists essentially of a housekeeping epitope if the sequence has immunogenicity that is comparable to a mature epitope while also having other residues that either promote or do not hinder its presentation in mature form.

Note that the following discussion sets forth the inventors' understanding of the operation of the invention. However, it is not intended that this discussion limit the patent to any particular theory of operation not set forth in the claims.

Epitope-Encoding Vector Constructs

The present invention provides nucleic acid constructs for use as therapeutic vaccines. The constructs include a coding region having a sequence that encodes a polypeptide. The polypeptide is an epitope of a TAA. In one embodiment, the target cell is a neoplastic cell and the polypeptide is an epitope or precursor of an epitope of a TuAA. In another embodiment, the target cell is any cell infected with an intracellular parasite. The term “parasite” as used herein includes any organism or infective agent such as a virus that has an intracellular stage of infection within the host. These include but are not limited to: viruses such as adenovirus, cytomegalovirus, Epstein-Barr virus, herpes simplex virus 1, herpes simplex virus 2, human herpesvirus 6, varicella-zoster virus, hepatitis B virus, hepatitis D virus, papilloma virus, parvovirus B19, polyomavirus BK, polyomavirus JC, hepatitis C virus, measles virus, rubella virus, human immunodeficiency virus (HIV), human T cell leukemia virus I, and human T cell leukemia virus II; bacteria such as Chlamydia, Listeria, Salmonella, Legionella, Brucella, Coxiella, Rickettsia, Mycobacterium; and protozoa such as Leishmania, Trypanasoma, Toxoplasma, and Plasmodium.

The polypeptide(s) encoded by the nucleic acid construct can include a housekeeping epitope of a TAA. In preferred embodiments, the nucleic acid construct encodes a plurality of housekeeping epitopes. When the construct encodes such a plurality, the multiple epitopes can all correspond to different segments of a single TAA, or they can correspond to different TAAs. In a preferred embodiment, the nucleic acid construct contains a housekeeping epitope and an immune epitope. In another preferred embodiment, the nucleic acid construct contains a housekeeping epitope and an epitope cluster region.

In preferred embodiments, wherein the construct of the vaccine encodes both a housekeeping epitope and an immune epitope, the vaccine can stimulate a cellular immune response against target cells presenting either epitope—that is, the immune response can recognize the housekeeping epitopes displayed initially by the target cells, and then can also recognize the immune epitopes presented by the target cells after induction by IFN.

Advantageously, the nucleic acid construct can further include a third or fourth sequence, or more, with such sequences encoding a third or fourth epitope, or additional epitopes, respectively. Such epitopes can be derived from a single TAA or from two or more different TAAs, and can be housekeeping or immune epitopes in any combination. The constructs can be designed to encode epitopes corresponding to any other proteasome activities that may play a role in processing antigens in any target cell or pAPC.

The encoded MHC epitopes are preferably about 7-15 amino acids in length, and more preferably, 9 or 10 amino acids in length. While the generally preferred peptide size for MHC I binding is 9 amino acids, shorter and longer peptides may also in some cases bind MHC I. Likewise, many peptides much longer than 9 amino acids can be trimmed by exopeptidases or other proteases resident in the cell, to produce fragments that bind MHC I very effectively. The size of a peptide containing an immune epitope sequence is not critical, so long as the sequence includes the epitope. This is because the immune proteasome, resident in the pAPC, in combination with trimming exopeptidases and other proteases, in its normal function correctly processes full length TAAs to produce immune epitopes. Thus, the nucleic acid sequence encoding the immune epitope can actually encode a much larger precursor, including the complete TAA. Such a construct preferably also encodes a housekeeping epitope.

Efficient discovery of effective TAA epitopes is discussed in detail in copending U.S. patent application Ser. No. 09/561,074 entitled “METHOD OF EPITOPE DISCOVERY,” filed on Apr. 28, 2002, which is incorporated herein by reference in its entirety.

Examples of TuAAs and other TAAs suitable for use in the present invention include but are not limited to: differentiation antigens such as MelanA (MART-I), gp100 (Pmel 17), tyrosinase, TRP-1, TRP-2, and tumor-specific multilineage antigens such as MAGE-1, MAGE-3, BAGE, GAGE-1, GAGE-2, CEA, RAGE, NY-ESO, SCP-1, Hom/Mel-40 and PRAME. Similarly, TuAAs include overexpressed oncogenes, and mutated tumor-suppressor genes such as p53, H-Ras and HER-2/neu. Additionally, unique TuAAs resulting from chromosomal translocations such as BCR-ABL, E2A-PRL, H4-RET, IGH-IGK, MYL-RAR and viral antigens such as Epstein Barr virus antigens EBNA, and the human papillomavirus (HPV) antigens E6 and E7 are included. Other useful protein antigens include butare not limited to TSP-180, MAGE-4, MAGE-5, MAGE-6, RAGE, NY-ESO, p185erbB2, p180erbB-3, c-met, nm-23H1, PSA, TAG-72-4, CAM 17.1, NuMa, K-ras, β-Catenin, CDK4, Mum-1, and p16. These and other TuAAs and pathogen-related antigens are known and available to those of skill in the art in the literature or commercially.

In a further embodiment, the TAA is an antigen specific for a virus. See Table 1. In yet another embodiment of the present invention, the TAA is an antigen specific for a non-viral intracellular parasite. Examples of parasite-specific antigens include nucleotides, proteins, or other gene products associated with the intracellular parasite. Suitable nucleotides or proteins can be found at the NCBI Taxonomy Database located at the internet hypertext transfer protocol on the world wide web, “ncbi.nlm.nih.gov/Taxonomy/tax.html/.” More detailed descriptions of gene products for parasites and other pathogens are provided at this web site.

TABLE 1

| Virus | Candidate Gene Products |
|---|---|
| Herpes Simplex I | ICP4, VP16, ICPO, γ1^{34.5} , g13 |
| EBV | ZTA, EBNA-2, EBNA-1, LMP-1, LMP-2, |
| | LMP-2a, LMP-2b |
| | EBNA-3, EBNA-4, EBNA-LP, EBNA- |
| | 3A, 3C, BZLF-1 |
| Poxvirus | VeTF, K3L, p37, A14L, A13L, A17L, |
| | A18R |
| SV40 | Large T antigen, Small T antigen, VPZ |
| Adenovirus | E1A, E3L, E1B, E4 (OEF6), E4 (ORF1), |
| | gp19K, ADP, RIDα, RIDβ |
| Hepatitis B | pX, L(pre-Si) |
| Htlv- 1 | Tax |
| HIV | TAT, GAG, MA, ENV, TM, NEF, VIF, |
| | VPR, REV, VPX |
| Hepatis B | NS5A |
| Reovirus | δ-3 |
| Rous Sarcoma Virus | pp60^{src} |
| Harvey Sarcoma Virus | p21^{ras} |
| HPV | E6, E7, E5 |
| Polyomavirus | LT, mT, sT |

Particularly preferred peptides are about 7-15 amino acids in length. An extensive listing of peptides having MHC binding motifs is provided in Han-Georg Rammensee, Jutta Bachmann, and Stefan Stevanovic, “MHC Ligands and Peptide Motifs,” Springer-Verlag, Germany, (1997) Landes Bioscience, Austin, Tex.

The epitopes encoded by the constructs have affinity to one or more MHC I alleles. In some embodiments, wherein a patient is heterozygous for MHC I, the construct can encode epitopes corresponding to different MHC I alleles.

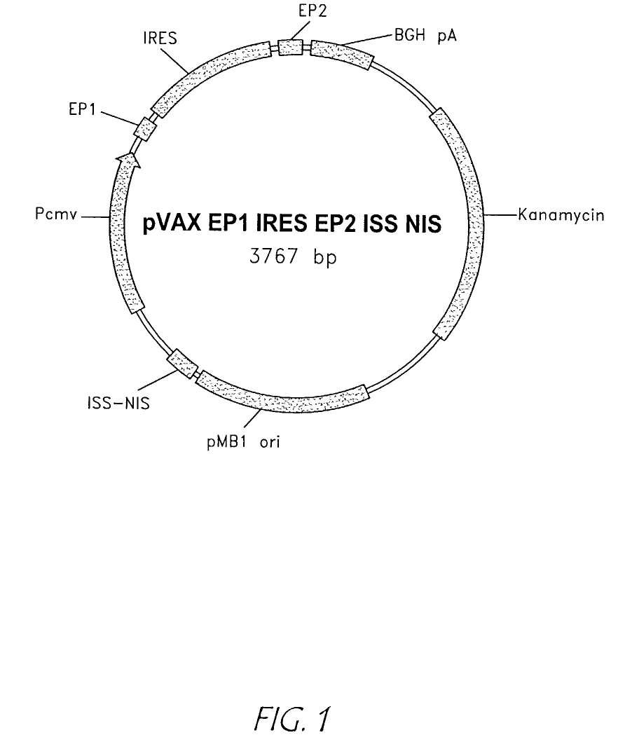

Preferred nucleic acid constructs include at least one promoter sequence that is operably linked to the 5′ end of the coding region of the construct. It will be appreciated by those of skill in the art that any promoter active in mammalian cells can be employed. Preferred promoter sequences include, but are not limited to, the CMV promoter, the SV40 promoter, and retroviral LTR promoter sequences, and can also include EF-1A, UbC, β-actin promoters. In some embodiments, the constructs can include two or more promoters that are operably linked to the 5′ end of different polypeptide-encoding sequences. Likewise, the constructs can employ enhancers, nuclear import sequences, immunostimulatory sequences, and expression cassettes for cytokines, selection markers, reporter molecules, and the like. Moreover, immunostimulatory, or other modulatory sequences can be attached to the vector via a stably hybridized peptide nucleic acid (PNA). In preferred embodiments, the nucleic acid constructs of the present invention also include a poly-A sequence that is operably linked to a 3′ end of the coding region. A nucleic acid construct that includes a nuclear import sequence and an immunostimulatory sequence is depicted in FIG. 1.

In certain embodiments, the nucleic acid constructs encode an mRNA that is translated as a single polypeptide and then cleaved. In one such embodiment the polypeptide consists of a linear array of epitopes, wherein the first (N-terminal) sequence is one or more immune epitopes or epitope clusters, and the second (C-terminal) sequence is a housekeeping epitope, such that the correct C-terminus of the housekeeping epitope is specified by the termination codon, and all other HLA epitope termini are determined by proteasomal processing and exopeptidase trimming.

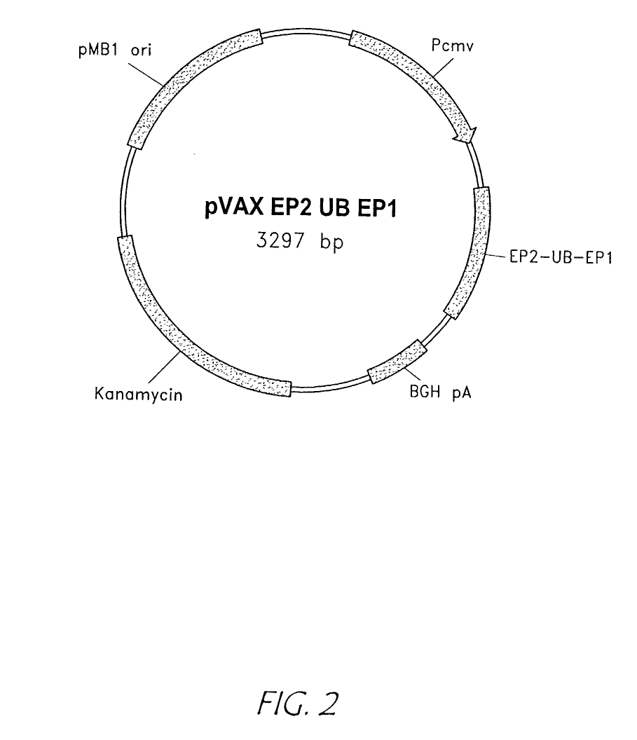

In another preferred embodiment, the nucleic acid construct encodes an amino acid sequence wherein an immune epitope or an epitope cluster is linked to a ubiquitin sequence. The ubiquitin sequence is similarly linked to a housekeeping epitope. The presence of ubiquitin between the epitopes facilitates efficient delivery of the immune epitope to the proteasome for epitope processing. The ubiquitin sequence (with or without an N-terminal spacer to ensure the integrity of the preceding peptide) is located in frame between the first and second sequence, or between any other epitope-encoding sequences. The so produced Sequencel-Ubiquitin-Sequence2 polypeptide is rapidly (co-translationally) cleaved at the Ubiquitin-Sequence2 junction by Ubiquitin-specific processing proteases, producing Sequence1-Ubiquitin and Sequence2. (See FIG. 2)

Physiologically, ubiquitin serves primarily as a signal that targets protein for degradation by the proteasome. It is among the most conserved proteins in eukaryotes, with only three conservative amino acid substitutions between yeast and human. Although the precise sequence of ubiquitin may vary somewhat, the sequence of a preferred embodiment is represented by SEQ ID NO: 2 (Ozkaynak, E., Finley, D., Solomon, M. J. and Varshavsky, A., The yeast ubiquitin genes: a family of natural gene fusions. EMBO J. 6 (5), 1429-1439 (1987)). Ubiquitin is a 76 amino acid long polypeptide having two crucial features: 1) a C-terminal Gly residue, involved in the conjugation of ubiquitin to the Lys side chain of protein substrates and 2) a Lys residue, at position 48, for the formation of multi-ubiquitin chains.

Ubiquitin genes are unique in the sense that all of them are synthesized as fusions to other polypeptides, including other ubiquitins. In the yeast S. cerevisiae, four ubiquitin genes have been identified: whereas the first three (UBI1-3) are fused to ribosomal proteins, the fourth gene (UBI4) is synthesized as a fusion of five identical repeats of the ubiquitin sequence. Thus, functional free ubiquitin is naturally produced after co-translational proteolytic processing by ubiquitously expressed ubiquitin-specific hydrolases. Such a natural organization has been exploited by generating C-terminal fusions between a single ubiquitin moiety and any desired polypeptide.

One has to distinguish between two conformations of ubiquitin: the first one is described above and consists of a linear fusion of a single ubiquitin to any desired polypeptide, in which the C-terminal Gly of ubiquitin is linked, via a peptide bond to the N-terminal amino acid of the polypeptide of choice. The second involves the conjugation of a ubiquitin moiety to a protein substrate, via a Gly-Lys bond formation. In this case, the COOH group of the ubiqutin Gly is linked to the ε (epsilon) side chain of a solvent exposed Lys of the substrate (or another ubiquitin moiety). It is only in the second case that ubiquitin exerts its signal for the degradation of the substrate. Thus, in the Sequence1-Ubiquitin-Sequence2 construct described above, Sequence2 is not targeted to the proteasome. Accordingly, the Sequence2 position is preferably used for a fully processed epitope, or one needing only N-terminal trimming, typically a housekeeping epitope. The ubiquitin moiety remaining attached to Sequence1 in the construct described above can be polyubiquitinated at Lys48, thereby targeting that fragment to the proteasome for processing, and resulting in the liberation of the epitope contained in Sequence1. It should be noted that if more than two sequences are linked together in a linear array by ubiquitin moieties, only the last sequence would behave in the manner of Sequence2; the processing of all of the upstream sequences would resemble that of Sequence1. To the extent that the constructs described herein are expressed in pAPCs, wherein only the immune proteasome is active, correct expression of housekeeping epitopes by these constructs benefits from the housekeeping epitopes being in the Sequence2 position, or a correspond position wherein the epitope does not require proteasomal processing in the pAPC.

In yet another embodiment the nucleic acid constructs of the present invention may include autoproteolytic peptide-encoding sequences. Such sequences are located between the first and second sequences or between any other epitope-encoding sequences. Examples of such autoproteolytic sequences include the inteins; also included are the 3C^{pro} and 2A^{pro} proteases of picornaviruses, including polioviruses and other enteroviruses, rhinoviruses, cardioviruses, and apthoviruses, and the equivalent cornoviridae proteases. These proteases catalyze the post-translational cleavage of the large precursor polyprotein made by this family of viruses.

In one embodiment, the autocatalytic protein sequence is inserted between two or more epitopes. In a further embodiment, the sequence is inserted after two or more epitopes but the cleavage signal is found between the epitopes such that they are cleaved into two or more fully functional epitopes. The type of protease is not important, it is only important that the appropriate cleavage signal be available for the correct processing of the epitopes.

Because the cleavage sites and the sequences of the autocatalytic proteins are known (recently reviewed by Seipelt, J. et al., Virus Research 62:159-168, 1999, which is incorporated herein by reference in its entirety) they can easily be used for construction of a vector which produces a polyprotein or biprotein. Briefly, 3C^{pro} predominantly recognizes a Q-G site as a cleavage signal although other closely adjacent positions can be important. Also the 3C^{pro} of some of these viruses adhere less closely to this general pattern, providing for a greater degree of flexibility in design. The limitation imposed by these requirements is more formal than real, particularly if the protease is placed between the epitopes to be expressed. In this arrangement an upstream immune epitope can be liberated by proteasomal processing even if the viral protease fails to cleave its N-terminus. The most crucial residues for cleavage at the C-terminus are internal to 3C^{pro} itself, generally leaving just 1-4 residues, if any, to be removed by exopeptidase trimming from the N-terminus of a downstream housekeeping epitope. 2A^{pro} can be used much the same way with the understanding that the cleavage site, while favoring G-P, is somewhat more variable among these viruses. It must also be considered that its expression can lead to a shutdown of host cell protein synthesis with a rapidity and completeness that depend on the virus strain from which it was derived.

Strictly speaking, the 2A proteins from cardioviruses and apthoviruses (i.e., Foot-and-Mouth Disease Virus (FMDV)) are not proteases, but rather prevent peptide bond formation at their C-termini without causing a termination of translation (Ryan, M. D., et al., Bioorganic Chemistry 27:55-79, 1999). Thus by positioning these 2A proteins between epitopes one can cause scission within a single reading frame. The 2A protein from FMDV is very small, only 18 amino acids, making it particularly well suited to multiple epitope expression. A plasmid employing the 2A protein is depicted as FIG. 3.

In certain other embodiments, the nucleic acid constructs encode an mRNA that is translated as two or more polypeptides. In one such embodiment the transcript can contain one or more internal ribosome entry site (IRES) sequences that are located between the first and second sequence or between any other epitope-encoding sequences. IRES sequences are naturally used by picornaviruses to direct internal cap-independent translation of mRNA. Such IRES sequences can also allow independent translation of two or more consecutive open reading frames from the same messenger RNA. Although the IRES sequences of various constructs may vary, the IRES sequence of one preferred embodiment is provided in SEQ ID NO:1 (Clontech PT3266-5). The C-terminus of each epitope expressed is determined by termination codons. Thus the order of the sequences encoding the housekeeping epitope and the sequences encoding the immune epitope does not matter, which provides flexibility of plasmid construction. Optionally, the sequence encoding the housekeeping epitope can precede the IRES sequence and the sequence encoding the immune epitope can be linked to the other end of the IRES sequence. Such vectors can also usefully encode two or more housekeeping epitopes. They can further allow the combination of the various single polypeptide constructs described above, in order to productively express multiple epitopes. See FIG. 4.

In certain other embodiments, the nucleic acid constructs encode two or more mRNA transcripts. Each of these transcripts may encode single epitopes or any of the dual or multiple epitope transcripts described in the embodiments above. Two or more transcripts can be the result of using multiple promoters. Those of skill in the art will recognize that use of more than one copy of a single promoter can lead to instability of the plasmid during propagation. Thus it will generally be preferable to use two (or more) different promoters.

Two or more transcripts can also be the result of using bidirectional promoters. Bidirectional promoters can be found in a wide variety of organisms. Examples of such promoters include PDGF-A from human, pcbAB and pcbC from Penicillium chrysogenum, neurotropic JC virus, and BRCA1 from mouse, dog and human. Although intensive research on bidirectional promoters began comparatively recently, there is a growing body of information on the sequence, regulation, and other intricacies of how they work. For example, the human transcobalamin II promoter requires a 69 base pair (bp) fragment containing a GC box and an E box for full transcriptional activity. The dipeptidylpeptidase IV promoter was shown to stimulate transcription from both sides with a similar efficiency. Rat mitochondrial chaperonins 60 and 10 are linked head to head and share a bidirectional promoter. Accordingly, various working bidirectional promoters have been identified, sequenced, and cloned in such a way that they can be used in a nucleic acid construct to express two genes.

Thus, in a preferred embodiment, the nucleic acid constructs contain bidirectional promoters such as, for example, those listed above, linked to a nucleic acid sequence encoding a housekeeping epitope or precursor thereof. In a particularly preferred embodiment, the nucleic acid construct contains bidirectional promoters linked to nucleic acid sequences encoding a plurality of housekeeping epitopes. In another embodiment, the nucleic acid constructs comprise bidirectional promoters linked to nucleic acid sequences encoding a housekeeping epitope and an immune epitope, or to an epitope cluster region. In addition, the bidirectional promoter may be positively or negatively regulated.

When the nucleic acid construct contains more than one epitope, the bidirectional promoter may express the plurality of epitopes in comparable amounts or some may be expressed at higher levels than the others. Alternatively, one epitope can be inducible and the other constitutive. In this way, a temporal regulation of epitope expression can be achieved, wherein one epitope is expressed early in the treatment and the other expressed later.

The compounds and methods described herein are effective in any context wherein a target cell displays housekeeping epitopes. Methods of discovering effective epitopes for use in connection with this invention are disclosed in copending U.S. patent application Ser. No. 09/561,074 entitled “METHOD OF EPITOPE DISCOVERY,” filed on Apr. 28, 2000, which is incorporated herein by reference in its entirety. Epitope synchronization technology and vaccines for use in connection with this invention are disclosed in copending U.S. patent application Ser. No. 09/560,465 entitled “EPITOPE SYNCHRONIZATION IN ANTIGEN PRESENTING CELLS,” filed on Apr. 28, 2000, which as mentioned above, was incorporated herein by reference in its entirety. Epitope clusters for use in connection with this invention are disclosed in copending U.S. patent application Ser. No. 09/561,571 entitled “EPITOPE CLUSTERS,” filed on Apr. 28, 2000, which is incorporated herein by reference in its entirety.

### EXAMPLES

### Example 1

### Construction of pVAX-EP1-IRES-EP2

Overview: The starting plasmid for this construct is pVAX1 purchased from Invitrogen.(Carlsbad, Calif.) Epitope EP1 and EP2 were synthesized by GIBCO BRL (Rockville, Md.). IRES was cut out from pIRES purchased from Clontech (Palo Alto, Calif.). See FIG. 4.

Procedure:

1 Digest pIRES with EcoRI and NotI. Separate the digested fragments with agarose gel, and purify the IRES fragment by gel purification;

2 Digest pVAX1 with EcoRI and NotI. Gel-purify the pVAX1 fragment;

3 Set up a ligation containing the purified pVAX1 and IRES fragment;

4 Transform competent DH5α with the ligation mixture;

5 Pick up 4 colonies and make a miniprep.

6 Perform restriction enzyme digestion analysis of the miniprep DNA. One recombinant colony having the IRES insert was used for further insertion of EP1 and EP2. This intermediate construct was called pVAX-IRES.

7 Synthesize EP1 and EP2;

8 Subclone EPI into pVAX-IRES between AflII and EcoRI site, to make pVAX-EP1-IRES;

9 Subclone EP2 into pVAX-EP1-IRES between SalI and NotI, to make the final construct pVAX-EP1-IRES-EP2;

10 Sequence the EP1-IRES-EP2 insert to confirm sequence.

### Example 2

### Construction of pVAX-EP1-IRES-EP2-ISS-NIS

Overview: The starting plasmid for this construct was pVAX-EP1-IRES-EP2 (Example 1). ISS (immunostimulatory sequence) introduced to this construct is AACGTT (SEQ ID NO. 4; Sato Y, Roman M, Tighe H, Lee D, Corr M, Nguyen M, Silverman G J, Lotz M, Carson D A and Raz E, Immunostimulatory DNA sequences necessary for effective intradermal gene immunization. Science, 273: 352-354 (1996)), and the NIS (standing for nuclear import sequence; SEQ ID NO. 3; Dean DA, Dean BS, Muller S, Smith L C, Sequence requirements for plasmid nuclear import. Exp. Cell Res. 253 (2): 713-22 (1999)) used is the SV4072 bp repeat sequence. ISS-NIS was synthesized by GIBCO BRL. See FIG. 1.

Procedure:

1 Digest pVAX-EP1-IRES-EP2 with NruI. Gel-purify the linearized plasmid;

2 Synthesize ISS-NIS;

3 Set up a ligation reaction containing the purified linearized pVAX-EP1-IRES-EP2 and synthesized ISS-NIS;

4 Transform competent DH5a with the ligation product;

5 Pick up colonies and make a miniprep;

6 Carry out restriction enzyme digestion of the miniprep;

7 Sequence the plasmid with the insert.

### Example 3

### Construction of pVAX-EP2-UB-EP1

Overview: The starting plasmid for this construct is pVAX1 (Invitrogen). EP2 and EPI were synthesized by GIBCO BRL. Wild type Ubiquitin cDNA encoding the 76 amino acids in the construct was cloned from yeast. See FIG. 2.

Procedure:

1 Perform RT-PCR using yeast mRNA. Primers were designed to amplify the complete coding sequence of yeast Ubiquitin;

2 Analyze the RT-PCR products using agarose gel. Gel-purify the band with the predicted size;

3 Subclone the purified DNA band into pZERO1 at EcoRV site. The resulting clone was named pZERO-UB;

4 Sequence several clones of pZERO-UB. Confirm Ubiquitin sequence before further manipulations;

5 Synthesize EP1 and EP2;

6 Ligate EP2, Ubiquitin and EPI and clone the insert into pVAX1 between BamHI and EcoRI, making it under control of the CMV promoter;

7 Confirm the sequence of the insert EP2-UB-EP1 by sequencing.

### Example 4

### Construction of a Tyrosinase Epitope Expressing Vector

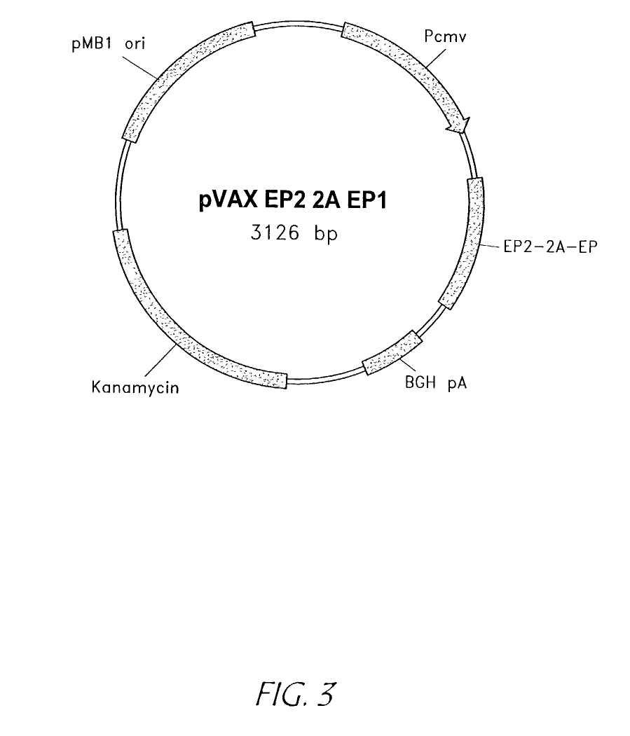

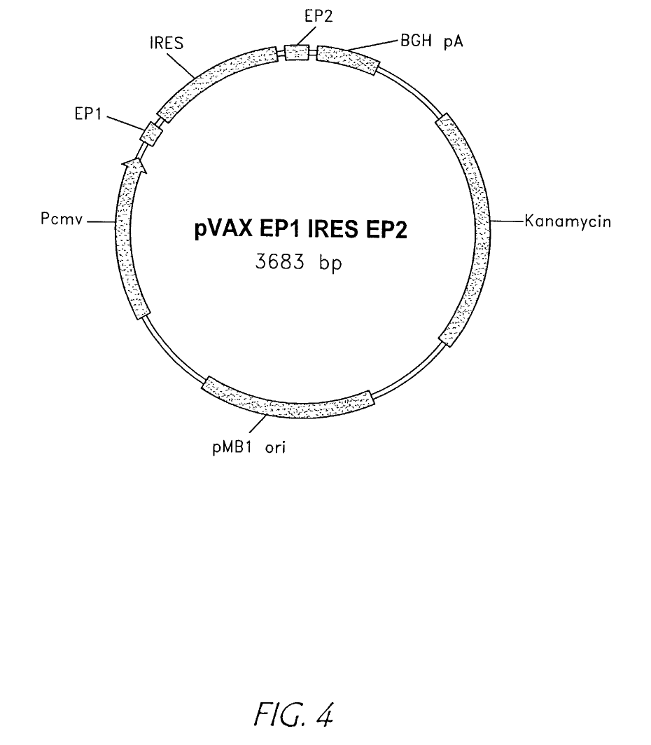

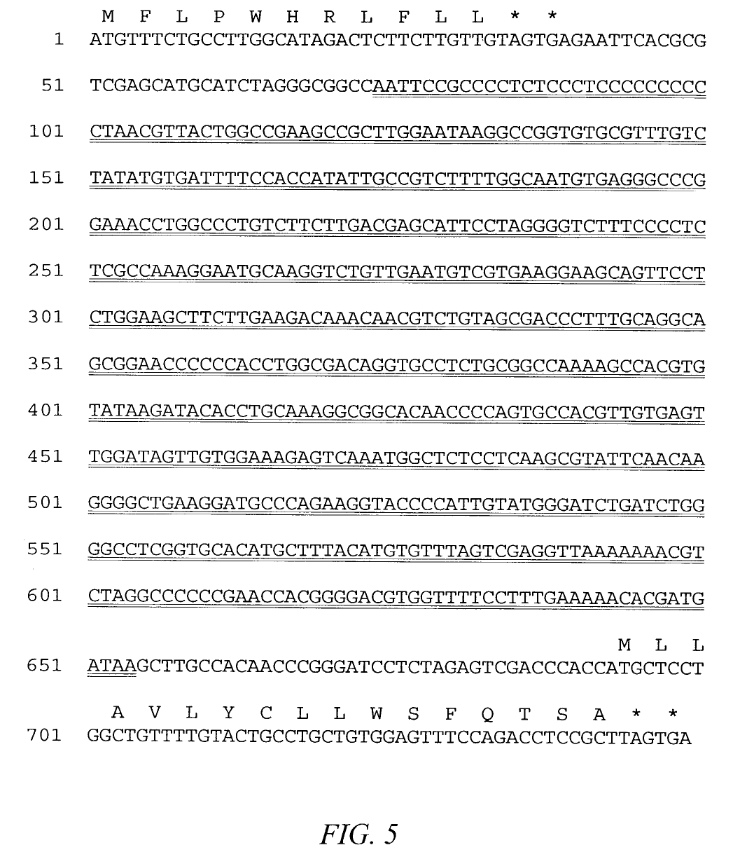

Construction of the vector was carried out as described in Examples 1 and 2 above. EPI encoded the housekeeping epitope Tyr₂₀₇₋₂₁₆, FLPWHRLFLL (SEQ ID NO. 5) (Entrez Protein Database Accession No. P14679) with an initiator methionine appended at the N-terminus MFLPWHRLFLL (SEQ ID NO. 6) and EP2 encoded the epitope cluster Tyr₁₋₁₇, MLLAVLYCLLWSFQTSA (SEQ ID NO. 7) (Entrez Protein Database Accession No. P14679). This is a SYNCHROTOPE™ vector. TA2M (SEQ ID NO. 8) is the immunogen-encoding sequences of this vector with the connecting IRES. FIG. 5 displays SEQ ID NO. 8 with the translations for the two encoded polypeptides, SEQ ID NOS. 6 and 7, shown above the DNA sequence in single letter amino acid code. The IRES, SEQ ID NO. 1, is double underlined. Positioning the initiator codon of SEQ ID NO. 7 in closer proximity or at the natural initiation position of the IRES, that is with a single T between the end of SEQ ID NO. 7 and the initiator codon, can constitute functionally similar sequences.

In constructing the polynucleotides encoding the polypeptides of the invention, the gene sequence of tyrosinase can be used, or the polynucleotide can be assembled from any combination of synonymous codons. Generally, for a 10 amino acid epitope this can constitute on the order of 10⁶ different sequences, depending on the particular amino acid composition. While large, this is a distinct and readily definable set representing a miniscule fraction of the >10¹⁸ possible polynucleotides of this length. Thus in some embodiments, equivalents encoding a particular sequence disclosed herein encompass such distinct and readily definable variations encoding the listed sequence. In choosing a particular one of these sequences to use in a vaccine or other composition, considerations such as codon usage, self-complementarity, restriction sites, chemical stability, etc. can be used as will be apparent to one skilled in the art.

It will also be apparent to one of skill in the art that amino acid sequence variants of SEQ ID NOS. 5-7, with functional or substantial similarity, can also be useful immunogens. N-terminal additions to housekeeping epitopes can be made with a great degree of freedom, although it is know that prolines can interfere with N-terminal trimming. Internal sequence variants of FLPWHRLFLL are constructed as follows. Consistent with the binding coefficient table (see Table 2) from the NIH/BIMAS MHC binding prediction program ((internet hypertext transfer protocol access at bimas.dcrt.nih.gov/molbio/hla_bin), described in Parker, K. C., et al., J. Immunol. 152:163, 1994; which is hereby incorporated by reference in its entirety), binding can be improved by changing the L at position 10, an anchor position, to V. Binding can also be altered, though generally to a lesser extent, by changes at non-anchor positions. Referring generally to Table 2, binding can be increased by employing residues with relatively larger coefficients. Changes in sequence can also alter immunogenicity independently of their effect on binding to MHC. Thus binding and/or immunogenicity can be improved as follows:

By substituting Y and W for F at position 1; these are equally preferred for binding and can provoke a useful cross-reactivity.

By substituting F, L, M, W, or Y for P at position 3; these are all bulkier residues that can also improve immunogenicity independent of the effect on binding. The amine and hydroxyl-bearing residues, Q and N; and S and T; respectively, can also provoke a stronger, cross-reactive response.

By substituting D or E for W at position 4 to improve binding; this addition of a negative charge can also make the epitope more immunogenic, while in some cases reducing cross-reactivity with the natural epitope. Alternatively the conservative substitutions of F or Y can provoke a cross-reactive response.

By substituting F for H at position 5 or the R at position 6 to improve binding H can be viewed as partially charged, thus in-some cases the loss of charge can hinder cross-reactivity. Substitution of the fully charged residues R or K at this position can enhance immunogenicity without disrupting charge-dependent cross-reactivity.

By substituting I, M, or V for L at position 7 to vary sequence without altering binding.

By substituting W for the F at position 8 to improve binding; this addition of a bulkier side-chain can also improve immunogenicity independent of the effect on binding. Substitution of D, E, K, R, H, M, S, T, Q, or N at this position are not generally predicted to reduce binding affinity by this model (the NIH algorithm), yet can be advantageous as discussed above.

By substituting F, W, or Y for the L at position 9 to improve binding; this addition of a bulkier side-chain can also improve immunogenicity independent of the effect on binding.

Finally, while substitutions in the direction of bulkiness generally improve immunogenicity, the substitution of smaller residues such as A, S, and C, at positions 3-9 can be useful according to the theory that contrast in size, rather than bulkiness per se, is a factor in immunogenicity. The reactivity of the thiol group in C can introduce other properties as discussed in Chen, J.-L., et al. J Immunol. 165:948-955, 2000, incorporated herein by reference in its entirety.

TABLE 2
9-mer Coefficient Table for HLA-A*0201*
HLA Coefficient table for file “A_0201_standard”

| Amino Acid | | | | | | | | | |
|---|---|---|---|---|---|---|---|---|---|
| Type | 1st | 2nd | 3rd | 4th | 5th | 6th | 7th | 8th | 9th |
| A | 1.000 | 1.000 | 1.000 | 1.000 | 1.000 | 1.000 | 1.000 | 1.000 | 1.000 |
| C | 1.000 | 0.470 | 1.000 | 1.000 | 1.000 | 1.000 | 1.000 | 1.000 | 1.000 |
| D | 0.075 | 0.100 | 0.400 | 4.100 | 1.000 | 1.000 | 0.490 | 1.000 | 0.003 |
| E | 0.075 | 1.400 | 0.064 | 4.100 | 1.000 | 1.000 | 0.490 | 1.000 | 0.003 |
| F | 4.600 | 0.050 | 3.700 | 1.000 | 3.800 | 1.900 | 5.800 | 5.500 | 0.015 |
| G | 1.000 | 0.470 | 1.000 | 1.000 | 1.000 | 1.000 | 0.130 | 1.000 | 0.015 |
| H | 0.034 | 0.050 | 1.000 | 1.000 | 1.000 | 1.000 | 1.000 | 1.000 | 0.015 |
| I | 1.700 | 9.900 | 1.000 | 1.000 | 1.000 | 2.300 | 1.000 | 0.410 | 2.100 |
| K | 3.500 | 0.100 | 0.035 | 1.000 | 1.000 | 1.000 | 1.000 | 1.000 | 0.003 |
| L | 1.700 | 72.000 | 3.700 | 1.000 | 1.000 | 2.300 | 1.000 | 1.000 | 4.300 |
| M | 1.700 | 52.000 | 3.700 | 1.000 | 1.000 | 2.300 | 1.000 | 1.000 | 1.000 |
| N | 1.000 | 0.470 | 1.000 | 1.000 | 1.000 | 1.000 | 1.000 | 1.000 | 0.015 |
| P | 0.022 | 0.470 | 1.000 | 1.000 | 1.000 | 1.000 | 1.000 | 1.000 | 0.003 |
| Q | 1.000 | 7.300 | 1.000 | 1.000 | 1.000 | 1.000 | 1.000 | 1.000 | 0.003 |
| R | 1.000 | 0.010 | 0.076 | 1.000 | 1.000 | 1.000 | 0.200 | 1.000 | 0.003 |
| S | 1.000 | 0.470 | 1.000 | 1.000 | 1.000 | 1.000 | 1.000 | 1.000 | 0.015 |
| T | 1.000 | 1.000 | 1.000 | 1.000 | 1.000 | 1.000 | 1.000 | 1.000 | 1.500 |
| V | 1.700 | 6.300 | 1.000 | 1.000 | 1.000 | 2.300 | 1.000 | 0.410 | 14.000 |
| W | 4.600 | 0.010 | 8.300 | 1.000 | 1.000 | 1.700 | 7.500 | 5.500 | 0.015 |
| Y | 4.600 | 0.010 | 3.200 | 1.000 | 1.000 | 1.500 | 1.000 | 5.500 | 0.015 |

*This table and other comparable data that are publicly available are useful in designing epitope variants and in determining whether a particular variant is substantially similar, or is functionally similar. When applied to a 10-mer, no calculation is performed for the 5^{th} amino acid of the sequence and the coefficients in the 5^{th} through 9^{th} columns are applied to the 6^{th} through 10^{th} amino acids.

### Example 5

### Design of Phase I Clinical Trial Using the SYNCHROTOPE™ TA2M Vaccine

Plasmid DNA vaccine encoding epitopes from tyrosinase was continuously infused intra-lymph nodally over 96 hours. Three cohorts of 8 patients each received increasing doses of plasmid. The lymph node was thus exposed to a high level of DNA in order to transfect local dendritic cells for effective presentation of encoded epitopes to T cells in the parafollicular areas. The toxicities and tolerability of the regimen were assessed, as well as the practicality of repeated cannulations of a groin lymph node for infusions. Immunologic and clinical responses were also measured.

Eligibility and Exclusion:

All patients in the study had histologically confirmed stage 1V melanoma by the modified 1988 AJCCIUICC staging system with an expected survival time of more than three months. Each patient was ambulatory with an ECOG performance status of 0 or 1. All patients were positive for HLA-A2. Patients were at least 18 years of age and agreed to use an acceptable method of birth control such as an intrauterine device, oral hormonal contraception, combination of spermicide and a barrier method, or abstinence during treatment. Female patients of childbearing potential had a confirmed negative urine pregnancy test on Day 0. All patients or their legally acceptable representative were required to comprehend and sign an informed consent approved by the National Institutes of Health Office of Biotechnology Activities and the Institutional Review Board at each site. This trial was conducted under a U.S. Food and Drug Administration Investigational New Drug Application IND BB 9146.

Patients were required to have neutrophils greater than 1500/μL, leukocytes greater than 3000/1L, platelets greater than 75,000/μL, and hemoglobin greater than 8.0 g/dL. Patients were excluded for hepatic disease as evidenced by AST or ALT>2.5×the upper limit of institutional normal, alkaline phosphatase >2.5×the upper limit of normal, or bilirubin>1.5×the upper limit of normal. Positive hepatitis B surface antigen or hepatitis C antibody and known or suspected renal impairment as evidenced by serum creatinine>1.5×the upper limit of normal or serum urea>2.6×the upper limit of normal were also exclusion criteria. Patients with ocular melanoma, history of brain metastases unless completely resected or a positive HIV test were also excluded.

Plasmid DNA:

A SYNCHROTOPE type™ TA2M (CTL ImmunoTherapies Corp., Chatsworth, Calif.) vaccine, which is a is a recombinant plasmid DNA vaccine, encodes two peptides, tyrosinase (207-216) and tyrosinase (1-17-), both of which are derived from human tyrosinase. The TA2M vaccine vector consists of 3683 base pairs of DNA. Its half-life in human serum in vitro was shown to be less than 20 minutes. The final product was purified to GMP standards by ion exchange chromatography including a non-ionic detergent to remove endotoxin and was supplied as a clear, colorless solution in buffered saline.

Treatment:

Patients were assigned sequentially to cohorts of eight to receive escalating doses of TA2M plasmid DNA over four 96-hour infusion periods. Group #1 received 200 μg on days 0, 14, 28, and 42. Group #2 received 400 μg and Group #3 received 800 μg. There was no intra-patient dose escalation. The decision to progress to a subsequent dose level was based upon dose limiting toxicity observed in two or fewer patients in the cohort by day 28 (two weeks following the second injection of plasmid on day 14).

Administration:

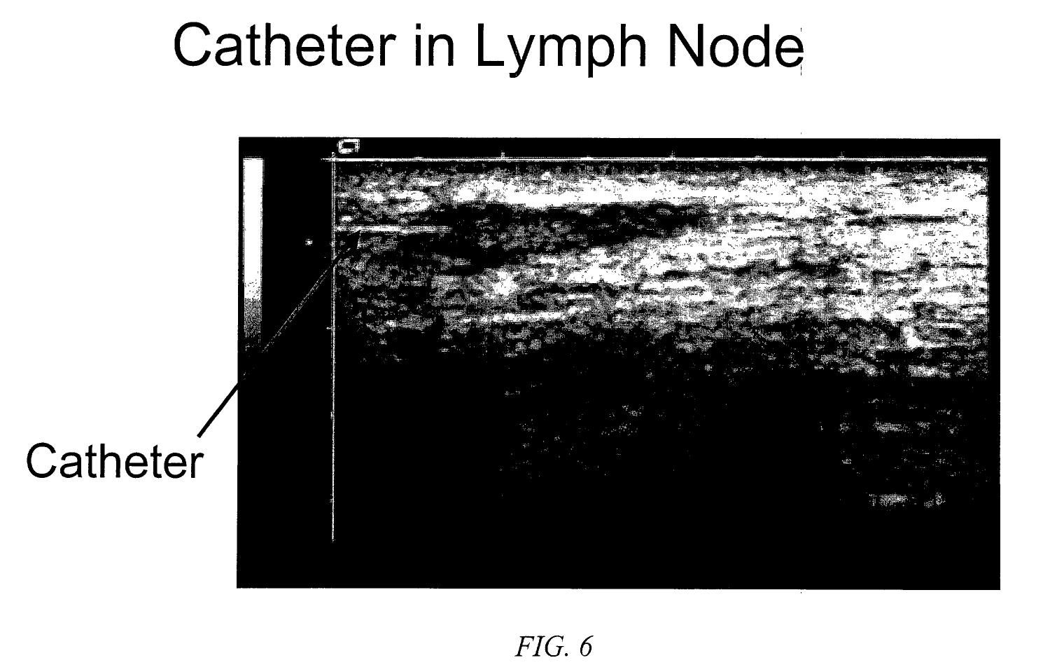

Plasmid DNA was administered via an infusion set (Silhouette™ Infusion set, Minimed Inc., Sylmar, Calif.) and portable pump (407C, Minimed Inc., Sylmar, Calif.) which had been approved by the FDA for the continuous delivery of medication. The plasmid DNA was delivered into a lateral superficial inguinal lymph node. These nodes were chosen for their relatively long major axes (1 to 2 cm) and because they are not adjacent to any major blood vessels. Using ultrasound (ATL HDI 5000, Phillips Ultrasound, Bothell, Wash.) with a high frequency linear array transducer, the infusion set was inserted into the long axis of the lymph node as indicated in FIG. 6. The 31 mm assembly consisted of a 23 gauge inner steel mandarin for stiffness and an outer 25 gauge plastic catheter. After insertion into the lymph node, the steel mandarin introducer was removed, and the system was fixed in place using an adhesive patch attached to the infusion set at the skin surface. On day 2, ultrasonographic evaluation was performed to confirm catheter placement. The presence or absence of extranodal fluid was noted, and if present, the catheter was assumed to be out of position. Patients were assessed at each visit for local adverse events including pain, swelling, and/or signs of infection.

Toxicity:

The TA2M plasmid DNA treatment was to be discontinued for any drug-related grade II allergic reaction, grade III non-hematologic toxicity, or any grade IV toxicity in a given patient. For grade II injection site pain, lymphedema, or phlebitis that occurred during an intranodal infusion, the dose was to be reduced by 50% for subsequent treatments; further grade II injection site pain, lymphedema, or phlebitis that occurred during an intranodal injection was to necessitate another 50% dose reduction. A third occurrence of grade II injection site pain, lymphedema, or phlebitis occurring in the same patient during an intranodal injection was to result in discontinuation of DNA plasmid administration.

Tetramer Immune Assay:

A quantitative assay using MHC class I-peptide tetramers was performed to estimate the magnitude of antigen-specific CD8+CTL among peripheral blood mononuclear cells. Assays were completed pre-study and after each 96-hour infusion cycle. An “immune response” was defined as at least a 2-fold increase in tetramer percentage after treatment or an increase to greater than 0.01%, which was regarded as the lower limit of detection for the assay. The tetramers containing the tyrosinase 207-216, tyrosinase 1-9, and tyrosinase 8-17 peptides were produced following the method of Altman (Altman J, Science 274:94-96, 1998; U.S. Pat. No. 5,635,363, which is hereby incorporated by reference in its entirety). Briefly, the plasmids encoding the extracellular domain on the HLA-A*0201 heavy chain fused to a biotinylation site, and full length human 132-microglobulin, were expressed as inclusion bodies in E. coli. Insoluble HLA-A*0201 and beta-2 microglobulin were dissolved in 8M Urea and refolded in the presence of tyrosinase peptides, then purified by gel filtration (FPLC). The product was biotinylated in the presence of 15 mg BirA (Avidity, Boulder, Colo.), 80 mM biotin, 10 mM ATP, 10 mM MgOAc, 20 mM bicine, and 10 mM Tris-HCl, pH 8.3. To remove free biotin, monomeric complexes were then purified by anion exchange (MonoQ), tested for biotinylation efficiency, and tetramerized by addition of Phycoerythrin (PE)-labeled strepavidin (Molecular Probes) at a 4:1 ratio. Tetramers were stored at 1-2 mg/mL at 4 degree Centigrade. The optimal concentration for each tetramer was validated and titrated using a HLA-A*0201 CTL clone specific for the appropriate peptide. Tetrameric assessment of CTL was accomplished by three color staining using Fluorescein Isothiocyanate (FITC) labeled anti-CD8, PerCP labeled anti-CD14/19 and PE labeled melanoma peptide or irrelevant control tetramer. CD8+ and CD14/19-lymphocytes were analyzed for PE labeling (tetramers binding) using a FACScan (Becton Dickinson, Mountain View, Calif.). The proportion of CD8+ cells that stained with tetramer was measured prior to and after vaccination, as described above.

DTH skin testing:

Delayed-type hypersensitivity was measured by intradermal injection of 100 μg of tyrosinase peptide 207-216 (SEQ ID NO. 5) produced by Multiple Peptide Systems, San Diego, Calif. Reactions were read after 24 hours. Indurations of 5 mm or more were considered positive.

Vector Containment and Systemic Plasmid Absorption:

The DNA manufacturing procedure for the plasmid vector fell under the Appendix C-II Escherichia Coli K-12 Host Vector Systems exemption according to NIH Guidelines, but guidelines for Biological Safety Level-1 were followed, as recommended by NIH. Based on nearly negligible potential toxicity for any component in the formulated DNA vaccine, specific decontamination procedures were not determined to be necessary. Soap and water cleaning was employed as needed.

Polymerase chain reaction (PCR) was performed to detect the presence of plasmid DNA in serum peripheral blood pre-study, on the first day of each infusion cycle, and on day 56. Quantitative detection of the specific nucleic acid sequences was performed using the fluorogenic 5′ nuclease assay.

Statistics:

Overall survival curves were constructed with the Kaplan-Meier method (Kaplan E, and Meier P. J Am Stat Assoc 53:457-481, 1958, which is hereby incorporated by reference in its entirety) using all evaluable patients based on the date of first administration of the vaccine. The overall comparison of the Kaplan-Meier curves was determined using the log-rank and Wilcoxon test. The log-rank tests were censored-data generalizations of the Savage (exponential scores) test and the Wilcoxon test using a large-sample chi-square test providing an overall comparison of Kaplan-Meier curves.

### Example 6

### Clinical Trial Demographics

Twenty six patients with stage 1V melanoma were treated in this trial and were evaluable for clinical and immune responses, since they received at least two doses of vaccine. The complete demographic data for all evaluable patients is listed in Table 3. There were 18 men and 8 women enrolled with a median age of 61 years (range 25-85). All patients were Caucasian. In 24 of 26 patients the primary site of diagnosis was the skin. Fourteen patients had measurable metastatic pulmonary disease (the most common measurable site of disease). Fourteen patients had a history of previous treatment with IL-2, chemotherapy, and/or biochemotherapy, 9 patients were previously treated with adjuvant high-dose interferon, and 8 patients had previously received some other form of vaccine therapy.

TABLE 3
Demographics

| | Dose | | | | | |
|---|---|---|---|---|---|---|
| Patient | (μg) | Gender | Age | Primary lesion | Pre-Strudy Target Lesion Site(s) | Prior Treatment |
| 001001 | 200 | F | 50 | R Parotid | Lung nodule x6, R neck mass | Dendritic Cell Vaccine with |
| | | | | | | radiotherapy |
| 001002 | 200 | M | 49 | R Calf | Scalp lesion, Liver lesions x6 | Biochemotherapy, radiotherapy |
| 001003 | 200 | M | 77 | Occipital scalp | Scalp x5 | Radiotherapy |
| 001004 | 200 | M | 69 | L posterior | Pancreatic mass | Interferon, CVD chemo, |
| | | | | auricular | | radiotherapy, vaccine |
| 001005 | 200 | M | 61 | scalp | Lung nodule x7 | Interferon, biochemotherapy, |
| | | | | | | antiangiogenesis agent |
| 001006 | 200 | M | 33 | vertex of scalp | Lung nodule x2, R pleural base, paragastric | Radiotherapy, |
| | | | | | | IL-12, biochemotherapy |
| 004002 | 200 | M | 45 | Right upper back | | Biochemotherapy, radiotherapy |
| 004003 | 200 | M | 69 | midchest | Axilla | Alpha interferon, vaccine |
| 001007 | 400 | F | 85 | scalp | Lung nodule x2 | Dacarbazine, tamoxifen |
| 001008 | 400 | M | 61 | L shoulder | Lung nodule x3 | Chemobiotherapy, interferon alpha |
| 001009 | 400 | M | 80 | L ear lobe | Lung nodule | peptide vaccine with IL-12 |
| 002001 | 400 | F | 48 | L cheek | Breast x2 | IL-2, gp 100 peptide vaccine, |
| | | | | | | interferon |
| 002002 | 400 | M | 69 | scalp | Lung nodule x3, spleen | High dose IFN, peptide vaccine |
| 002003 | 400 | M | 75 | L cheek | Lung x3, pleura | High dose IL-2 |
| 002004 | 400 | M | 55 | Axillary node | Multiple skin sites | High dose IL-2 + peptide |
| 002005 | 400 | F | 81 | | Knee | Radiotherapy |
| 004004 | 400 | M | 65 | left elbow | Lung | Biochemotherapy |
| 001010 | 800 | M | 63 | L neck | Lung mass x2 | Peptide vaccine with GM-CSF |
| 001011 | 800 | M | 67 | R neck | Adrenal glands bilaterally, spleen, | Alpha interferon, radiotherapy |
| | | | | | mesentery, vertebral mass | |
| 001012 | 800 | M | 47 | L auricle | Lung nodule, Jaw | Cancer vaccine w/GM-CSF, dendritic |
| | | | | | | cells |
| 001013 | 800 | F | 63 | R Mediastinal | Pleura x2, Lung nodule x4, Liver x4 | Radiation, apomine, CVD |
| | | | | | | chemotherapy, interferon alpha |
| 001014 | 800 | F | 25 | R upper back | Liver x2, subcutaneous nodule | Chemobiotherapy |
| 002006 | 800 | F | 39 | skin | Multiple skin sites, mediastinal lymph nodes | IL-2, gp 100 peptide vaccine |
| 004006 | 800 | M | 39 | rght thigh | Lymph nodes x3 | Interferon alpha |
| 004007 | 800 | F | 53 | right vulva | Right groin | none |
| 004008 | 800 | M | 49 | skin | Mesenteric lymph node | Interferon alpha |

### Example 7

### Toxicities

Toxicity from the TA2M vaccine was minimal. The overall toxicities and adverse events are listed in Table 4. There were no dose-limiting toxicities noted as a result of any of the 107 infusions. Only 2 of 27 patients had any dose-modifying toxicity (one patient in the 800 μg cohort required a reduction to 400 μg, and one patient required a reduction from 200 μg to 100 μg during a second four-infusion course). The most common toxicities and adverse events of administration were related to local pain, swelling, and/or redness either at the infusion site or lymph nodes (16 definite or probable reports in 10 patients, including one patient receiving a second cycle of plasmid infusion). Eight of the 16 reports related to local symptoms occurred in five patients at the 200 μg dose, 3 reports in two patients at the 400 μg dose, and 5 reports in three patients at the 800 μg dose. There was one grade I allergic reaction noted at the 800 μg dose. There were no reported hematologic manifestations of infusion. Systemic symptoms such as fever or fatigue were minimal (two patients at the highest dose reported fatigue).

### Example 8

### Immune Responses

Tetramer Assay

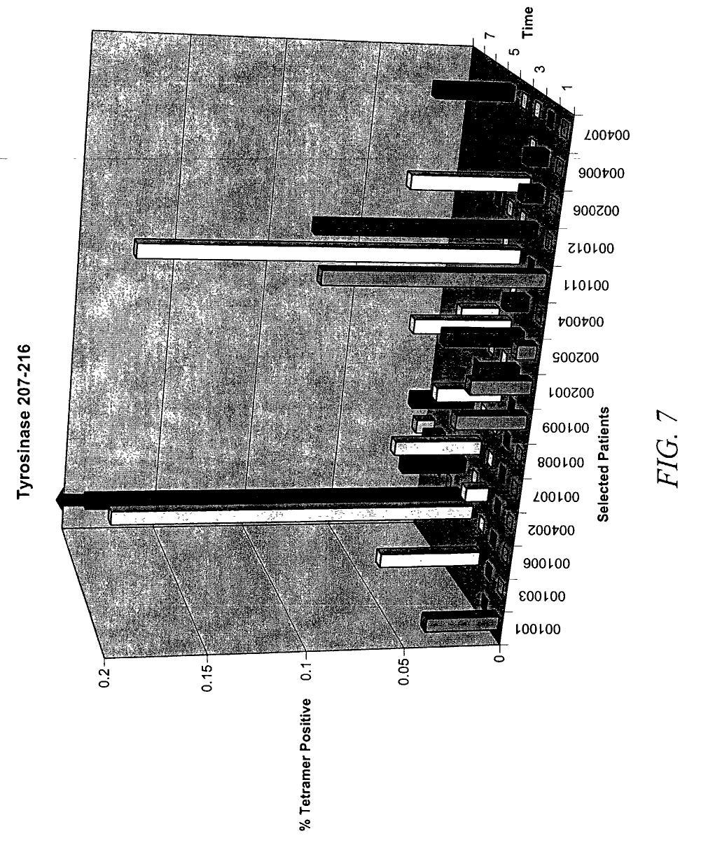

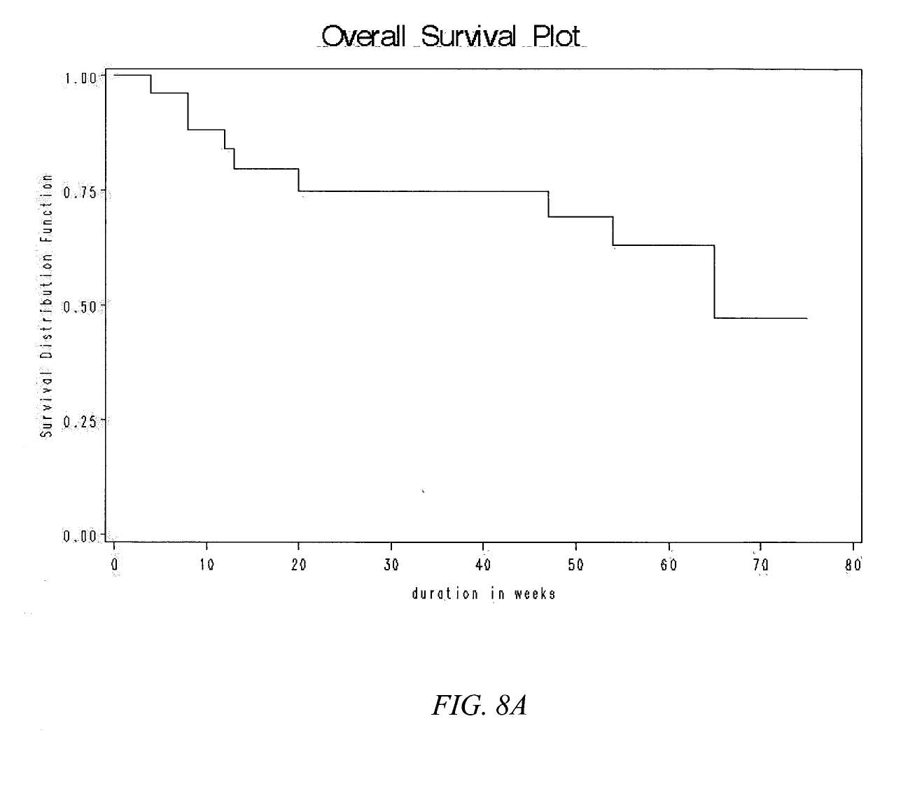

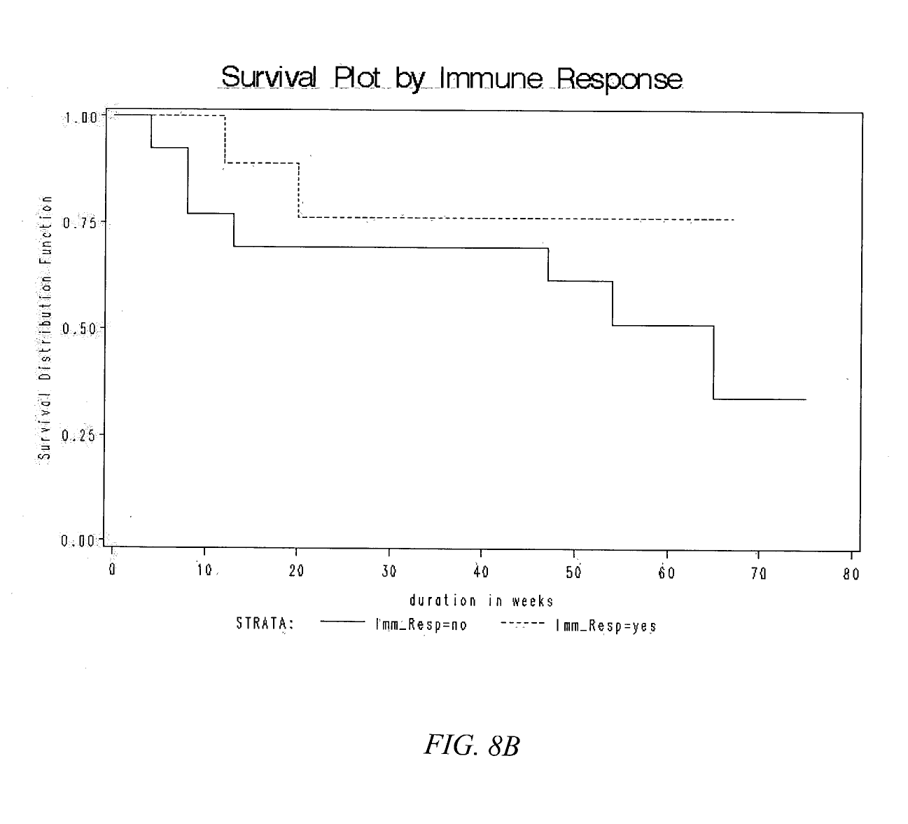

Immune response to tyrosinase 207-216 as demonstrated by tetramer assay is shown in FIG. 7. There were no or minimal responses to tyrosinase 1-9 or 8-17 by tetramer assay. Fifteen of the 24 immunologically evaluable patients had a positive tetramer assay (any detectable tetramer positivity greater than 0.01%) as shown in FIG. 7. The remaining nine patients demonstrated no or minimal tetramer positivity, defined as fewer than 1:10,000 CD8+ cells detected by flow cytometry. Of the 15 of 24 evaluable patients with a positive tetramer assay, four received 200 μg of TA2M, six received 400 μg, and five received 800 μg. One patient, #001006, at the 200 μg level showed a marked increase in tetramer positivity (0.75% positivity, which is out of the range in FIG. 7). Two of the clinically evaluable patients, #001013 and #001014, did not have tetramer assay performed and thus are not included in the figure.

Eleven of the 15 patients with detectable tetramer positivity demonstrated an immune response, defined as an increase in tetramer positivity greater than 0.01% from a baseline of undetectable or at least a two-fold increase in tetramer positivity over time. The remaining four tetramer positive patients had baseline positivity, decreasing tetramer assays over the course of the study, or the increase in their tetramer assays was less than two-fold. These four patients were not counted as immunological responders.

DTH Skin Test Results

Skin testing with intradermal injection of tyrosinase peptide 207-216 was positive in 6 of 24 patients immunologically evaluable patients. Of the 6 patients with positive DTH hypersensitivity skin tests, 2 received 200 μg, 1 received 400 μg, and 3 received 800 μg of SYNCHROTOPE™ TA2M. Five of the six patients with positive DTH tests were also positive by the tetramer assay.

### Example 9

### Detection of Serum Plasmid DNA by PCR

A PCR assay was performed to detect the presence of plasmid DNA in serum pre-study, on the first day of each infusion cycle, and on day 56. All samples except two were below the lower limit of detection, defined as less than 50 copies of plasmid per microgram of human genomic DNA. Two samples from patients receiving 800 μg of TA2M were positive, with levels of 71,882 copies/mL (patient #004006) and 1,256 copies/mL (patient #004008).

What is claimed is:

1. A nucleic acid construct comprising a first coding region, wherein the first coding region comprises a first sequence encoding a first polypeptide, wherein the first polypeptide consists essentially of a first housekeeping epitope corresponding to a housekeeping proteasome cleavage product of tyrosinase, wherein the mature housekeeping epitope is an MHC epitope having a sequence selected from the group consisting of SEQ ID NO. 5, a sequence with functional similarity to SEQ ID NO. 5, and a sequence with substantial similarity to SEQ ID NO. 5.

2. The nucleic acid construct of claim 1, wherein the MHC epitope has the sequence of SEQ ID NO. 5.

3. The nucleic acid construct of claim 1, wherein the first polypeptide has a sequence selected from the group consisting of SEQ ID NO. 5, SEQ ID NO. 6, a sequence with functional similarity to SEQ ID NO. 5, a sequence with functional similarity to SEQ ID NO. 6, a sequence with substantial similarity to SEQ ID, NO. 5, and a sequence with substantial similarity to SEQ ID NO. 6.

4. The nucleic acid construct of claim 3, wherein the first polypeptide has the sequence of SEQ ID NO. 5 or SEQ ID NO. 6.

5. A nucleic acid construct comprising a first coding region, wherein the first coding region comprises a first sequence encoding a first polypeptide, wherein the first polypeptide consists essentially of a first housekeeping epitope corresponding to a housekeeping proteasome cleavage product of a first antigen associated with a melanoma cell, wherein the mature housekeeping epitope is an MHC epitope, wherein the first coding region further comprises a second sequence encoding a second polypeptide, wherein the second polypeptide consists essentially of an epitope cluster derived from tyrosinase.

6. The nucleic acid construct of claim 5, wherein the first coding region and the second coding region are transcribed as segments of a single transcript, joined by an IRES.

7. The nucleic acid construct of claim 5, wherein the epitope cluster has a sequence selected from the group consisting of SEQ ID NO. 7, a sequence with functional similarity to SEQ ID NO. 7, and a sequence with substantial similarity to SEQ ID NO. 7.

8. The nucleic acid construct of claim 7, wherein the epitope cluster consists of the sequence of SEQ ID NO. 7.

9. The nucleic acid construct of claim 5, wherein the mature housekeeping epitope is an MHC epitope, the epitope having a sequence selected from the group consisting of SEQ ID NO. 5, a sequence with functional similarity to SEQ ID NO. 5, and a sequence with substantial similarity to SEQ ID NO. 5.

10. The nucleic acid of claim 9, wherein the MHC epitope consists of the sequence of SEQ ID NO. 5.

11. A nucleic acid construct comprising a sequence selected from the group consisting of SEQ ID NO. 8, a sequence with functional similarity to SEQ ID NO. 8, and a sequence with substantial similarity to SEQ ID NO. 8.

12. The nucleic acid construct of claim 11, comprising the sequence of SEQ ID NO. 8.

13. A nucleic acid construct comprising a first coding region, wherein the first coding region comprises a first sequence encoding a first polypeptide, wherein the first polypeptide consists essentially of a first housekeeping epitope corresponding to a housekeeping proteasome cleavage product of a first antigen associated with a melanoma cell, wherein the mature housekeeping epitope is an MHC epitope, further comprising a second coding region comprising a second sequence encoding a second polypeptide, wherein the second polypeptide consists essentially of an epitope cluster derived from tyrosinase.

14. The nucleic acid construct of claim 13, wherein the epitope cluster has a sequence selected from the group consisting of SEQ ID NO. 7, a sequence with functional similarity to SEQ ID NO. 7, and a sequence with substantial similarity to SEQ ID NO. 7.

15. The nucleic acid construct of claim 13, wherein the mature housekeeping epitope is an MHC epitope having a sequence selected from the group consisting of SEQ ID NO. 5, a sequence with functional similarity to SEQ ID NO. 5, and a sequence with substantial similarity to SEQ ID NO. 5.


SEQUENCE LISTING (8 sequences)

SEQ ID NO: 1 - DNA, 581 bp, Encephalomyocarditis Virus
aattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataa

SEQ ID NO: 2 - DNA, 228 bp, Saccharomyces cerevisiae
atgcagattttcgtcaagactttgaccggtaaaaccataacattggaagttgaatcttccgataccatcgacaacgttaagtcgaaaattcaagacaaggaaggtatccctccagatcaacaaagattgatctttgccggtaagcagctagaagacggtagaacgctgtctgattacaacattcagaaggagtccaccttacatcttgtgctaaggctaagaggtggc

SEQ ID NO: 3 - DNA, 72 bp, Artificial Sequence
  Synthetic Nucleotide Sequence
tggttgctgactaattgagatgcatgctttgcatacttctgcctgctggggagcctggggactttccacacc

SEQ ID NO: 4 - DNA, 6 bp, Artificial Sequence
  Synthetic Nucleotide Sequence
aacgtt

SEQ ID NO: 5 - protein, 10 aa, Homosapien
FLPWHRLFLL

SEQ ID NO: 6 - protein, 11 aa, Artificial Sequence
  Tyr207-216 with initiator methionine
MFLPWHRLFLL

SEQ ID NO: 7 - protein, 17 aa, Homosapien
MLLAVLYCLLWSFQTSA

SEQ ID NO: 8 - DNA, 749 bp, Artificial Sequence
  Tyr Immunogen-encoding sequences with connecting IRES
atgtttctgccttggcatagactcttcttgttgtagtgagaattcacgcgtcgagcatgcatctagggcggccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggatcctctagagtcgacccaccatgctcctggctgttttgtactgcctgctgtggagtttccagacctccgcttagtga